(19) United States
(12) Patent Application Publication
(10) Pub. No.: US 2022/0370363 A1
(43) Pub. Date: Nov. 24, 2022

(54) NOVEL METHOD OF POLYMERIC NANOPARTICLE FABRICATION FOR CANCER TREATMENT AND OTHER DRUG DELIVERY APPLICATIONS

(71) Applicant: HOWARD UNIVERSITY, Washington, DC (US)

(72) Inventor: Simeon K. ADESINA, Mitchellville, MD (US)

(73) Assignee: HOWARD UNIVERSITY, Washington, DC (US)

(21) Appl. No.: 17/535,752

(22) Filed: Nov. 26, 2021

Related U.S. Application Data
(60) Provisional application No. 63/118,078, filed on Nov. 25, 2020.

(51) Int. Cl. A61K 9/16

(52) U.S. Cl. CPC A61K 9/1694; A61K 9/1647

(57) ABSTRACT

A novel and innovative method of fabricating nanoparticles with reproducible characteristics from batch-to-batch and during scale-up. The method is a dipolymerization-precipitation reaction facilitated by the inverse electron demand Diels-Alder (IEDDA) reaction.

### CROSS-REFERENCE TO RELATED APPLICATIONS

This application claims benefit of U.S. Provisional Application No. 63/118,078 filed Nov. 25, 2020, the entire content of which is incorporated herein by reference.

### TECHNICAL FIELD

Methods pertain to novel fabricating of polymeric nanoparticles for cancer treatment and other drug delivery applications.

### BACKGROUND

In spite of the huge body of information and scientific research data available on the development of drug-loaded polymeric nanoparticles for cancer therapy, none is currently approved by the Food and Drug Administration (FDA) for use to treat cancer in the United States (Anselmo et al., “Nanoparticles in the clinic: An update. Bioengineering and Translational”, Medicine 2019; 4: e10143; Bhardwaj et al., “Recalcitrant Issues and New Frontiers in Nano-Pharmacology”, Frontiers in Pharmacology, 2019, 10:1369. doi: 10.3389/fphar.2019.01369). The fact that no polymeric nanoparticle formulation is approved by the FDA for clinical use in cancer chemotherapy, despite the fact that the commonly used polymers for nanoparticle fabrication, the polyesters, such as poly(lactide-co-glycolide) (PLGA) are FDA approved for clinical use in humans, shows that there are challenges associated with the development of these polymeric nanoparticles and consequently, their clinical use. The use of the polyesters so far have been restricted to the development of depot preparations and microspheres usually for other disease conditions but not for cytotoxic chemotherapeutic agents (Blasi, “Poly (lactic acid)/poly (lactic-co-glycolic acid)-based microparticles: an overview”, Journal of Pharmaceutical Investigation, 2019, 49:337-346).

The clinically used FDA-approved nanoparticle drug delivery systems for cancer treatment are mostly liposomal formulations with nanoparticle albumin-bound paclitaxel (Abraxane) being an exception (Anselmo et al. supra; Bhardwaj et al. supra). The only polymeric nanoformulation that has been approved so far for cancer therapy (Genexol-PM) was approved in Korea in 2006 (Lee et al., “An Open-Label, Randomized, Parallel, Phase II Trial to Evaluate the Efficacy and Safety of a Cremophor-Free Polymeric Micelle Formulation of Paclitaxel as First-Line Treatment for Ovarian Cancer: A Korean Gynecologic Oncology Group Study (KGOG-3021)”, Cancer Research and Treatment: Official Journal of Korean Cancer Association, 2018, 50(1): 195-203). The interest in the clinical use of polymeric nanoparticles stems from the fact that these systems are biocompatible and biodegradable, are more stable and have better control over release of encapsulated drugs compared to liposomes (Venkatraman et al., “Polymer- and liposome-based nanoparticles in targeted drug delivery”, Frontiers in Bioscience S2, 2010, 801-814).

Major challenges to the clinical use of polymeric nanoparticles from a drug delivery and targeting standpoint include poor reproducibility during fabrication and scale-up and poor targeting and efficacy in humans (Bhardwaj et al., supra). Several methods are used to prepare nanoparticles with varying degrees of success in achieving consistent nanoparticle characteristics (Rezvantalab et al., “PLGA-Based Nanoparticles in Cancer Treatment”, Frontiers in Pharmacology, 2018, 9:1260. doi: 10.3389/fphar.2018.01260). The properties of polymeric particles are strongly dependent on the preparation method (Swider et al., “Customizing poly (lactic-co-glycolic acid) particles for biomedical applications”, Acta Biomaterialia, 2018, 73:38-51). The technique used to produce polymeric nanoparticles can impact the size, drug loading and release characteristics. Batch-to-batch consistency in these key areas is important for cytotoxic drugs in the treatment of cancer (Streck et al., “Comparison of bulk and microfluidics methods for the formulation of polylactic-co-glycolic acid (PLGA) nanoparticles modified with cell-penetrating peptides of different architectures”, International Journal of Pharmaceutics: X1(2019) 100030. https://doi.org/10.1016/j.ijpx.2019.100030).

For polymer-based nanoparticles, challenges include the inability to consistently obtain particle sizes around 100 nm, maintain batch-to-batch consistency and maintain size uniformity (low polydispersity) which can cause differences in uptake and consequently drug efficacy (Streck et al., supra). Formulation scientists are trying to find new and different methods of nanoparticle preparation and modification to gain tight control over PLGA degradation, drug release, and other characteristics (Rezvantalab et al., “PLGA-Based Nanoparticles in Cancer Treatment”, Frontiers in Pharmacology, 2018, 9:1260. doi: 10.3389/fphar.2018.01260).

Overcoming the above identified challenges lead to more consistent results amongst batches, leading to clinical success. Therefore, an approach that will facilitate the fabrication of nanoparticles with reproducible characteristics from batch-to-batch and during scale-up is essential and necessary for clinical use.

### SUMMARY OF THE INVENTION

The present inventor has discovered a novel and innovative method of fabricating polymeric nanoparticles. This novel method will facilitate the preparation of nanoparticles with reproducible characteristics from batch-to-batch and during scale-up. The method described herein comprises a dipolymerization-precipitation nanoparticle preparation mechanism facilitated by inverse electron demand Diels-Alder (IEDDA) reaction. The described method is of commercial interest to pharmaceutical companies and companies involved in the development of nanotechnologies and drug delivery platforms for the treatment of cancers and other diseases.

### DETAILED DESCRIPTION OF THE INVENTION

The present method is a novel method to prepare nanoparticles to facilitate excellent batch-to-batch reproducibility during nanoparticle fabrication and scale-up using the inverse electron demand Diels-Alder (IEDDA) reaction to facilitate dipolymerization followed by nanoparticle precipitation in a mixture of solvents. Using this strategy, it is the solvent composition among other formulation variables at the point of polymer coupling that determines polymer precipitation and particle formation. In the present method, this variable can easily be controlled from batch to batch and with scale-up. Optimization of the nanoparticle formation process by varying formulation and process variables will lead to reproducible nanoparticle characteristics.

The present inventor has contributed to the use of dispersion polymerization for drug delivery applications (Adesina et al., “Optimization of the fabrication of novel stealth PLA-based nanoparticles by dispersion polymerization using D-optimal mixture design”, Drug Development and Industrial Pharmacy, 2013, 40(11): 1547-1556; Adesina et al., “Polylactide-based Paclitaxel-loaded Nanoparticles Fabricated by Dispersion Polymerization: Characterization, Evaluation in Cancer Cell Lines, and Preliminary Biodistribution Studies”, Journal of Pharmaceutical Sciences, 2014, 103(8): 2546-2555; Akala and Adesina, “Chapter 1—“Fabrication of polymeric core-shell nanostructures” Nanoscale Fabrication, Optimization, Scale-Up and Biological Aspects of Pharmaceutical Nanotechnology”, Pages 1-49, Edited by Alexandru Mihai Grumezescu. Elsevier Inc. (2018) ISBN: 978-0-12-813629-4; the content of all of which is incorporated herein by reference). In dispersion, the starting reaction mixture is a clear, single-phase solution; particles are formed by precipitation of growing polymer chains. Therefore, the solvent medium becomes a dispersion medium (Horak, “Effect of reaction parameters on the particle size in the dispersion polymerization of 2-Hydroxyethyl Methacrylate”, J. Polym. Sci. A Polym. Chem., 1999, 37: 3785-3792; Capek, “Surface active properties of polyoxyethylene macromonomers and their role in radical polymerization in disperse systems”, Adv. Colloid Interface Sci., 2000, 88(3): 295-357; Leobandung et al., “Monodisperse Nanoparticles of Poly(ethylene glycol) Macromers and N-Isopropyl Acrylamide for Biomedical Applications”, J. Appl. Polym. Sci., 2003, 87:1678-1684; Song et al., “Monodisperse, controlled micron-size dye-labeled polystyrene particles by two stage dispersion polymerization”, Polymer, 2006, 47: 817-825; Ha et al., “Size Control of Highly Monodisperse Polystyrene Particles by Modified Dispersion Polymerization”, Macromol. Res., 2010, 18(10): 935-943).

Nanoprecipitation requires the addition of two solvents that are miscible with each other and the method results in instantaneous formation of nanoparticles, is easy to perform, can be easily scaled up and is a one-step procedure (Yadav et al., “Modified Nanoprecipitation Method for Preparation of Cytarabine-Loaded PLGA Nanoparticles”, AAPS PharmSciTech., 2010, 11(3): 1456-1465; the content of which is incorporated herein by reference). In nanoprecipitation, polymer and drug are dissolved in a water miscible organic solvent, for example, acetone or methanol. The solution is then added into an aqueous solution which contains a surfactant in a drop-wise manner. Through rapid solvent diffusion, the nanoparticles are formed immediately. The solvent is then removed under reduced pressure (Wang et al., “Manufacturing Techniques and Surface Engineering of Polymer Based Nanoparticles for Targeted Drug Delivery to Cancer”, Nanomaterials, 2016, 6, 26; the content of which is incorporated herein by reference). The size uniformity of polymeric nanoparticles is the most significant parameter that decides the consistency of performance.

Nanoprecipitation yields particles with broad particle size distribution affecting the consistency of performance which affects clinical translation (Chidambaram et al., “Modifications to the Conventional Nanoprecipitation Technique: An Approach to Fabricate Narrow Sized Polymeric Nanoparticles”, Adv. Pharm Bull., 2014, 4(2): 205-208). Particles with broad particle size distribution leads to difficulty in establishing the size of particles responsible for the biological effects. With dispersion polymerization on the other hand, although the reaction belongs to solution polymerization before the stage of the nucleation, the polymerization and polymer particle growth occur within the particles and, consequently, it is difficult to obtain uniform particles and uniform size distribution. In addition, the requirement of low monomer concentration and/or special processes hinder the practical application of precipitation polymerizations and these requirements are essential to avoid the coagulation of the resultant polymer particles (Liu et al., “Self-Stabilized Precipitation Polymerization and Its Application”, Research, 2018, Volume 2018, Article ID 9370490, 12 pages). Chemical initiators used in dispersion polymerization may also confer toxicity.

To ensure particle size uniformity, reproducibility and facilitate clinical use of polymeric nanoparticle platforms, Inverse Electron Demand Diels-Alder (IEDDA) chemistry is used to facilitate dipolymerization followed by precipitation. The IEDDA reaction is used between dienes, e.g. tetrazines, and strained dienophiles (e.g. trans-cyclooctene, TCO) in a bioorthogonal reaction (Oliveira et al., “Inverse Electron Demand Diels-Alder Reactions in Chemical Biology”, Chem. Soc. Rev., 2017, 46, 4895-4950; the content of which is incorporated herein by reference). Other bioorthogonal reactions such as strain promoted alkyne-azide cycloaddition or other forms of click chemistry or any other polymer coupling approach using various polymer coupling chemistries process may be used to prepare nanoparticles). Thus, the “Adesina method” is a dipolymerization-precipitation method or polymer coupling method of nanoparticle preparation described herein. The Adesina method is the method of the invention. The [4+2] cycloaddition of 1,2,4,5-tetrazines and various dienophiles such as TCO is very fast (10,000 times faster than the copper-catalyzed click reaction), selective, biocompatible and catalyst-free (Oliveira et al., supra). It is safe and biocompatible that it can be used in vivo for radiolabeling using pretargeting methodologies. Because of its characteristics, IEDDA can proceed in low concentrations of reactants. Using IEDDA, various dienes such as tetrazines as a non-limiting example and dienophiles such as trans-cyclooctene and norbornenes, as non-limiting examples, can be mixed in suitable ratios to form conjugates. The reaction proceeds at rapid rates without the use of catalysts or any other additives in different solvents such as dichloromethane, acetonitrile, tetrahydrofuran, methanol and ethanol as non-limiting examples.

Polymers that can be used include the polyesters such as polylactide, polycaprolactone, polylactide-co-glycolide, copolymers of the polyesters with polyethylene glycol, other copolymers, or any other polymer or copolymer that can be functionalized to bear dienes and dienophiles to facilitate the IEDDA reaction or with other functional groups to facilitate polymer coupling. Using this approach, the choice of solvent used must be such that the different polymers are initially soluble in the solvent but upon conjugation, the dipolymer becomes insoluble in the solvent. A combination of solvents could also be used such that a non-solvent for the dipolymer (e.g. water, alcohols) may be combined with the solvent for the constituent polymers to facilitate nanoparticle precipitation.

As an example of the present method, commercially available HO-PLGA-PEG (HO-poly (lactide-co-glycolide)-polyethylene glycol) or HO-PLGA (HO-poly (lactide-co-glycolide) is functionalized with trans-cyclooctene (TCO; a dienophile) to yield TCO-PLGA-PEG or TCO-PLGA respectively. Another commercially available HO-PLGA is functionalized with tetrazines (such as methyltetrazine, MTZ) to yield Tetrazine-PLGA. The reaction of these functionalized PLGA molecules (TCO-PLGA-PEG or TCO-PLGA and Tetrazine-PLGA) that is dissolved and soluble in a rationally selected combination of solvents leads to a polymer dimer with double the molecular weight and thus insoluble in the original solvent mixture. This leads to precipitation of the polymer and its assembly to form nanoparticles based on the composition of the initial solvent mixture. If there is water in the solvent mixture for example, the PEG molecules orient into the aqueous phase forming the corona of the nanoparticle while the PLGA forms the core leading to the formation of a PEG-coated stealth nanoparticle capable of prolonged circulation in blood. Since only a dimer is formed (unlike polydisperse polymers and oligomers in dispersion polymerization), relatively precise insoluble polymer chain lengths are formed which leads to relatively precise particle sizes with very narrow size distribution. Also, there is no particle growth as obtained in dispersion polymerization and other polymerization methods of nanoparticle fabrication leading to narrow particle size distribution. In addition, the PEG at the surface acts as a steric stabilizer and prevents coagulation of particles thereby controlling the particle size in addition to conferring stealth properties to the nanoparticle in vivo.

Other non-limiting examples of the present method is the reaction between TCO-PEG and MTZ-PLGA (Methyltetrazine-PLGA) or TCO-PLGA and MTZ-PEG of suitable molecular weights in a suitable solvent composition. Additional examples include the reaction between TCO-PLGA-PEG and MTZ-PLGA-PEG. The molecular weight of polymers to be functionalized with TCO-(trans-cyclooctene) or MTZ-(methyltetrazine), and the solvent composition for the reaction among other formulation variables are essential in this approach and must be optimized.

While the subject matter disclosed herein has been described in connection with what is presently considered to be practical example embodiments, it is to be understood that the present disclosure is not limited to the disclosed embodiments, and covers various modifications and equivalent arrangements included within the spirit and scope of the present invention.

1. A method of fabricating nanoparticles with reproducible characteristics, comprising a dipolymerization-precipitation reaction facilitated by the inverse electron demand Diels-Alder (IEDDA) reaction.

2. A method of fabricating nanoparticles with reproducible characteristics, comprising functionalizing HO-PLGA-PEG (HO-poly (lactide-co-glycolide)-polyethylene glycol) or HO-PLGA (HO-poly (lactide-co-glycolide) with trans-cyclooctene (TCO) to yield TCO-PLGA-PEG or TCO-PLGA, respectively, and
dissolving TCO-PLGA-PEG or TCO-PLGA in a solvent mixture to produce a polymer dimer with double the molecular weight and thus insoluble in the solvent mixture.

3. A method of fabricating nanoparticles with reproducible characteristics, comprising functionalizing HO-PLGA (HO-poly (lactide-co-glycolide) with tetrazine to yield Tetrazine-PLGA, and
dissolving Tetrazine-PLGA in a solvent mixture to produce a polymer dimer with double the molecular weight and thus insoluble in the solvent mixture.
